(19) United States
(12) Patent Application Publication
(10) Pub. No.: US 2010/0331530 A1
(43) Pub. Date: Dec. 30, 2010

(54) METHOD FOR PURIFYING THERAPEUTIC PROTEINS BY MEANS OF MULTI-STAGE EXTRACTION

(75) Inventors: Werner Bäcker, Wipperfurth (DE); Sven Sommerfeld, Schwelm (DE); Martina Mutter, Koln (DE); Paula Alexandra Albuquerque De Jesus Rosa, Lisboa (PT); Maria Raquel Murias Dos Santos Aires-Barosl, Lisboa (PT); Ana Margarida Nunes Da Mata Pires De Azevedo, Lisboa (PT)

Correspondence Address: GERSTENZANG, WILLIAM C.;NORRIS MCLAUGHLIN & MARCUS, PA, 875 THIRD AVE, 8TH FLOOR, NEW YORK, NY, 10022, US

(73) Assignee: BAYER TECHNOLOGY SERVICES GMBH, LEVERKUSEN (DE)

(21) Appl. No.: 12/919,084

(22) PCT Filed: Feb. 25, 2009
(86) PCT No.: PCT/EP2009/001313

§ 371 Date: Aug. 24, 2010

(30) Foreign Application Priority Data
Mar. 10, 2008 (DE) 10 2008 013 490.2

(51) Int. Cl. C07K 1/16; C07K 1/14; C07K 1/34; C07K 1/30

(52) U.S. Cl. 530/413; 530/422; 530/414; 530/418

(57) ABSTRACT

Method for purifying therapeutic proteins by multi-stage extractive distillation.

The present invention relates to a method for purifying proteins, in particular therapeutic proteins, by means of multistage extraction.

Biological products are frequently purified by chromatographic methods. In these methods, usually separation mechanisms such as, e.g. ion exchange, hydrophobic interaction, affinity and size exclusion are utilized. In addition to the advantage of the high selectivity, these techniques, however, have a number of disadvantages such as, e.g. the complex packing of the columns, the low intraparticle diffusion, the high pressure drop over the packing, a low capacity, low chemical and proteolytic stability and the high costs of the adsorbent.

One alternative offered here is by aqueous 2-phase extraction (ATPE). It can be used at the same time for cell separation, concentration and first purification of the target component from complex mixtures such as fermentation broths or biological extracts [P. A. Albertsson, Partition of Cell Particles and Macromolecules, Wiley, New York, 1986; M. Rito-Palomares, J. Chromatogr., B807 (2004) 3] and is thus a robust purification method for a multiplicity of separation problems from mixtures containing biological material. The technical effect of these separation operations is based on mutual incompatibility of two polymers or of one polymer and one salt at certain concentrations. In addition to a very good biocompatibility owing to the high water content (80-90% (w/w) of water) and the low surface tension of these systems, in single-stage methods generally, a certain selectivity and yield can be achieved already by varying the experimental conditions (e.g. concentration, ph, ionic strength, molecular weight of the polymers) [P. A. Albertsson, Partition of Cell Particles and Macromolecules, Wiley, New York, 1986].

US 2007/0048786 discloses a method in which proteins are fractionated into classes by means of single-stage or multistage extraction or liquid-liquid separation. The extraction is, for example, an aqueous 2-phase extraction. The method refers to further analysis of the concentrated fractions. The object of providing individual proteins in a quality usable for therapeutic application is not disclosed. The resulting fractions and also the extraction method may therefore be described as coarse. There is therefore no disclosure with respect to instructions for obtaining proteins in pure form.

U.S. Pat. No. 4,728,613 discloses a method by means of which it is possible to obtain extracellular enzymes from beer fermentations. The method comprises mixing the entire fermentation broth with a polymer and an inorganic salt, which forms an aqueous 2-phase system. The sought-after enzymes accumulate in this case in the polymer phase. Usable polymers which are disclosed are polyethylene glycols, amines of polyethylene glycols, carboxylates of polyethylene glycols, polypropylene glycols, and also their amines and carboxylates, polyethylene glycol esters, polyethyleneimines, trimethylamino-polyethylene glycols, poly(vinyl alcohol)s, polyvinylpyrrolidones and mixtures thereof. The method comprises only one extraction step, and also optionally a separation of the enzyme from the aqueous polymer phase. The purity of the enzyme after it is obtained is only about 90-92%. In addition, the enzyme-rich phase is significantly contaminated with 5-20% by volume of the salt phase. Therefore, the method is unsuitable for producing high-purity proteins.

The problem of inadequate purity of the extraction product is addressed by the disclosure of U.S. Pat. No. 5,151,358, and also U.S. Pat. No. 5,139,943. Both pass the extraction products of a first aqueous 2-phase extraction through an ion-exchange column in order to obtain the target protein chymosin in purer form. Therefrom follows the requirement that the protein must be recovered from the column, which in turn is accompanied by the above-described disadvantages of chromatography.

U.S. Pat. No. 4,879,234 discloses a complex method in which formate dehydrogenase from Candida boidinii is obtained by subjecting cell material to two sequential extractions. In the first step, the cell material containing the formate dehydrogenase is exposed to a first 2-phase system, wherein the one phase comprises an aqueous solution of polyethylene glycol or polypropylene glycol and the other phase comprises an aqueous phase containing dextran, methyl cellulose or Ficoll as phase-forming agents, and also a triazine dye which is chemically bound to polyethylene glycol or polypropylene glycol. According to U.S. Pat. No. 4,879,234, after a certain time, the formate dehydrogenase is then situated in the upper phase. The lower phase of the first 2-phase system is discarded and an aqueous phosphate solution is added to the upper phase, whereby a second 2-phase system is formed again, wherein according to the disclosure the formate dehydrogenase then collects in the lower phase. This is separated off from the upper phase and the formate dehydrogenase is obtained therefrom, in turn, e.g. by means of ultrafiltration. The upper phase in the second step, comprising the triazine dye chemically bound to polyethylene glycol or polypropylene glycol, can be reused. The formation of the first 2-phase system is according to the disclosure an affinity separation system. The separation of the two phases can proceed in each case using industrial apparatuses such as, e.g., a nozzle separator or a disk separator.

U.S. Pat. No. 4,879,234 discloses a lyophilization as sole further preparation step after the above-described procedure. However, this is unsuitable for achieving a reliable separation of the extraction agents (in particular the chemically modified polyethylene glycol, or polypropylene glycol) which would be impermissible, in particular for therapeutic proteins. In addition, it is questionable to what extent the method is economically efficient if the modified polyethylene glycol or polypropylene glycol, which is certainly expensive in manufacture, cannot be recovered quantitatively. Quantitative recovery, however, is physically excluded, since in the context of extraction methods, parts of a phase are always carried over into another phase. Therefore, the method disclosed in U.S. Pat. No. 4,879,234 must be described as economically disadvantageous for application to therapeutic proteins.

U.S. Pat. No. 6,437,101 discloses the isolation of human growth hormone (HGH), a growth hormone antagonist or a mixture of the two from a biological source, using an aqueous 2-phase extraction. A two-stage extraction is likewise disclosed, wherein the phase resulting in the first extraction stage which is low in target protein is subjected to a further back extraction, whereas the target-protein-rich phase resulting from the first extraction stage is not subjected to a further extraction step. The back extraction therefore apparently serves for increasing the yield of the method. U.S. Pat. No. 6,437,101 discloses no further extraction of the target-protein-rich fraction for further increasing the purity. Therefore, the use of a back extraction cannot be understood as a further purification stage. Nevertheless, U.S. Pat. No. 6,437,101 discloses that the purity of proteins is of importance especially in the case of later application as therapeutic agent and refers to the prior art for further purification of the target protein from the respective target-protein-rich phases of the two extraction stages. These comprise, in particular, the above-described chromatographic methods, also precipitations, centrifugations or else gel electrophoresis. U.S. Pat. No. 6,437,101 excludes the use of chaotropic agents. Chaotropic agents comprise detergents, for example. Washing, e.g. in the form of a further extraction of the target-protein-rich phase for increasing the purity thereof is also not disclosed.

Since in many separations, however, more than one separation step is necessary in order to achieve the required yields and/or purities, it is a technical object to provide a method which makes it possible, in an economically advantageous manner, the simplest possible standard devices, in a multistaged construction such as, e.g., mixer-settler devices or extraction columns, to purify therapeutic proteins from mixtures with at least one high-molecular-weight impurity and at least one low-molecular-weight impurity.

It has surprisingly been found that a method for purifying therapeutic proteins starting from a mixture A, which method comprises at least one therapeutic protein (P), at least one high-molecular-weight impurity (H) and at least one low-molecular-weight impurity (N), characterized in that it comprises the steps:
a) mixing the mixture A with a phase A (PA), obtaining a solution 1,
b) extracting the solution 1 resulting from step a), using a phase B (PB), obtaining a solution b1 comprising a portion of the phase B (PB), at least a portion of the therapeutic protein (P) and at least a portion of the low-molecular-weight impurity (N), and also obtaining a solution b2 comprising a portion of the phase A (PA), at least a portion of the high-molecular-weight impurity (H) and optionally a portion of the therapeutic protein (P),
c) further extracting the solution b1 resulting from step b), using a phase C (PC), obtaining a solution c1, comprising at least a portion of the supplied phase C (PC), at least a portion of the therapeutic protein (P) and optionally a portion of the low-molecular-weight impurity (N), and also obtaining a solution c2, comprising at least a portion of the phase B (PB) supplied with solution b1, at least a portion of the low-molecular-weight impurity (N) and optionally a portion of the therapeutic protein (P),
d) further extracting the solution b2 resulting from step b) using further phase B (PB), obtaining a solution d1 comprising at least a portion of the phase A (PA) supplied with solution b2 and at least a portion of the high-molecular-weight impurity (H), and also obtaining a solution d2 comprising at least a portion of phase B (PB) and optionally a portion of the therapeutic protein (P),
e) washing the solution c1 resulting from step c) using further phase B (PB), obtaining the one solution e1, comprising at least a portion of the phase C (PC), at least a portion of the therapeutic protein (P), optionally a portion of the low-molecular-weight impurity (N), and also obtaining a solution e2, comprising at least a portion of the phase B (PB), at least a portion of the low-molecular-weight impurity (N) and optionally a portion of the therapeutic protein (P),
   is able to achieve this object.

In the context of the present invention, therapeutic proteins (P) designate all molecules which comprise a stringing together of amino acids and which are formed in the context of the biological activity of living organisms or are chemically identical to such molecules and can be used in medical procedures on mammals.

Preference is given to therapeutic proteins (P) which can be used in medical procedures on humans. Non-exhaustive examples are immunoglobulins (e.g. IgG, IgM, IgD), myoglobins and albumins.

Impurity, in the context of the present invention, designates any substance which comprises the mixture A which is not the therapeutic protein (P). Impurities can also comprise proteins which likewise have a therapeutic use according to the above definition that are merely not the target of the purification in the respective configuration of the present invention. Mixture A can also contain organisms or other solids.

The designations low-molecular-weight and high-molecular-weight are, in the context of this invention, to be considered as relative to the molecular mass of the therapeutic molecule. A low-molecular-weight impurity (N) therefore designates impurities of a lower molecular mass than that of the therapeutic protein (P), whereas high-molecular-weight impurity (H) comprises an impurity which has a higher molecular mass than the therapeutic protein (P).

Non-exhaustive examples of low-molecular-weight or high-molecular-weight impurities (H, N) are insulin, growth hormones, albumins, interleukins, interferons, DNA, lecitin, erythropoietin, glucose, lactate and/or amino acids. Whether these, in the specific case, are low-molecular-weight impurities (N) or high-molecular-weight impurities (H) depends, as already stated above, on the molecular mass of the therapeutic protein (P).

The phases A and C according to the invention (PA, PC) customarily comprise solutions of at least one salt and/or at least one polymer in water. If the phases A and/or C (PA, PC) are solutions comprising at least one salt in water, then the at least one salt can be composed of the cations selected from the list ammonium, lithium, sodium, potassium, cesium, magnesium, calcium, strontium, barium, iron, manganese and thiocyanate, and also anions selected from the list: borate, bromide, hydrogencarbonate, carbonate, chloride, citrate, fluoride, nitrate, nitrite, phosphate, monohydrogenphosphate, dihydrogenphosphate, sulfate, sulfite and 2-amino-2-(hydroxymethyl)propane-1,3-diolate (TRIS). Preferably, the cations are potassium and/or sodium. Likewise preferably, the anions are citrate, chloride, phosphate, monohydrogenphosphate, dihydrogenphosphate and/or sulfate. Particularly preferably the salts are sodium chloride, sodium dihydrogenphosphate, potassium hydrogenphosphate and/or sodium citrate.

In the individual steps of the method according to the invention, the salts can be used in differing portions and in differing mixtures in the phases A and C (PA, PC). The total portion of the salts in the phases A and C (PA, PC) is preferably between 0 and 40% by weight.

If the phases A and/or C are solutions comprising at least one salt in water, then phases A (PA) particularly preferably comprise sodium chloride in a portion of 0-20% by weight and phases C (PC) particularly preferably comprise less sodium chloride than phases A (PA).

The difference between the two phases A and C (PA, PC) in addition to the preferably differing portion of sodium chloride, lies especially in the fact that the phase A (PA) is a less good solvent for the therapeutic protein (P) than for the phase B (PB), whereas the phase C (PC) is a better solvent for the therapeutic protein (P) than the phase B (PB).

This difference in the solubility of the therapeutic protein (P) in phases A and C (PA, PC) is utilized in the further extraction according to step c) of the method according to the invention in order to purify further the therapeutic protein (P).

Furthermore, the phases A and C (PA, PC) are characterized in that they are essentially immiscible with the phase B (PB). Essentially in the context of the present invention means a portion less than 1% by weight.

The phases A and/or C (PA, PC) can have a higher or lower density than the phase B (PB). The density of the phases A and/or C and/or B can be set, e.g., via the portion and/or the type of the salts and/or polymers present therein. Preferably, at least the phase A (PA) has a higher density than the phase B (PB). Particularly preferably, the phases A and C (PA, PC) have a higher density than the phase B (PB).

If the phases A and C (PA, PC) are solutions comprising at least one polymer in water, then the at least one polymer can be a dextran or a starch or a starch derivative. Non-exhaustive examples of suitable starches or starch derivatives are, for instance, waxy barley starch (93-95% by weight amylopectin and 5-7% by weight amylase having a molecular weight of about 500 kDa) or hydroxypropyl starches, preferably having a molecular weight of about 100 kDa to about 200 kDa. Suitable starches and starch derivatives can be obtained, for example, under the trade names Reppal®PES100 or Reppal®PES200 from Lyckeby Culinar AB, Sweden.

The phase B (PB) according to the invention customarily comprises a solution of at least one polymer which is preferably soluble in water up to a portion of 10% by weight. Likewise preferably, the phase B (PB) also comprises salts such as are used in the phases A and/or C (PA, PC) according to the invention.

Preferred polymers are polyethylene glycols, polypropylene glycols and also derivatives of the two abovementioned polymers, block copolymers of these, poly(vinyl alcohol)s, poly(methyl alcohol)s, poly(ethyl alcohol)s, poly(ether alcohol)s, polyvinylpyrrolidones, polyacrylates, dextrans, starch derivatives as already described above, maltodextrans, cellulose derivatives.

Particular preference is given to polyethylene glycol.

Very particular preference is given to polyethylene glycol having molar masses between 400 and 22 000 g/mol. A particularly preferred phase B (PB) comprises 5-40% by weight of polyethylene glycol of a molar mass of about 3350 g/mol, and also sodium chloride, a hydrogenphosphate salt and a dihydrogenphosphate salt.

The polymers used can be thermosensitive, or not. In an alternative embodiment of the invention, thermosensitive polymers are used. Thermosensitive polymers, in the context of the present invention, are taken to mean polymers which lead to the aqueous solution in which they are dissolved separating, as a result of heating, into two phases of different density. One example of a thermosensitive polymer is, for instance, a block copolymer of polyethylene and polypropylene.

Extraction, in the context of the present invention, means a two-stage procedure in each case, wherein in a first step, two liquid phases are brought into contact with one another and, in a second step, the phases are separated again. The contacting of the phases can be carried out, for example, by stirring, by enforcing a turbulent flow, according to methods known to those skilled in the art, such as, for instance, passage through a gap or passing the two phases past one another in countercurrent.

Preference is given to a procedure in which the contact is achieved by passing the two phases past one another in countercurrent. A good mass transfer is thereby achieved between the two phases, without the later separation becoming too complex. The second step of the extraction can be achieved, for example, by the method of centrifugation known to those skilled in the art, but also simply by settling the two phases. The settling corresponds in this case to centrifugation in the earth's gravitational field. Preference is given to a simple settling of the phases, since this can keep the expenditure of the method in terms of equipment low.

Particularly preferably, each washing/each extraction is carried out in mixer-settler devices, comprising at least one extraction/wash zone with countercurrent flow and at least one settling zone.

In addition, it is characteristic of an extraction in the context of the present invention that transfer of the therapeutic protein (P) is carried out in an extraction from one phase of the aqueous 2-phase extraction system into the other phase.

Washing therefore, in contrast to the extraction according to the invention, designates the special case that the therapeutic protein (P) remains in the same phase before and after carrying out such a process step and instead an impurity (H, N) is transferred from one phase to another.

It is known to those skilled in the art that, neither in the case of extraction nor in the case of washing, is exact selection of the therapeutic protein (P) or of the impurity (H, N) possible. Rather, the two definitions of washing and extraction relate to any substance (impurity or therapeutic protein) which, owing to the favorable distribution coefficient in the 2-phase system, is preferentially transferred from one phase to the other phase.

The distribution coefficient is a matter-system-specific parameter and designates a quotient K, calculated from the concentration of a substance in a phase X divided by the concentration of the same substance in a phase Y at chemical equilibrium (i.e. after an infinitely long time) at a defined pressure and at a defined temperature.

It is also clear therefrom that a distribution coefficient likewise exists between the phases themselves, and so portions of a phase are always present in the other respective phase when these have been brought into contact. As described above, however, this is preferably the case only at a portion less than 1% by weight.

The possibilities are known to those skilled in the art by means of which the value of the distribution coefficient of the substances at least to be extracted/washed (H, P, N) can be changed. Non-exhaustive examples which can be used here are the change in the ratios of the salts optionally contained in the phases (PA, PB, PC, PD, PE, PF), the change in the temperature or else the change of pressure.

Furthermore, the methods are known to those skilled in the art by means of which they can achieve subsequently the portions of the respective extraction or wash phases (PA, PB, PC) in the solutions obtained from the extractions or from the washing. Non-exhaustive examples of such methods are setting sufficiently long residence times in settling zones in which the two phases are separated from one another, the use of suitable devices for supporting the separation (such as for instance centrifuges), and also setting the intensity of the mixing of the two phases before the respective separation, for instance by setting lower speeds of rotation of optionally used stirrers.

Suitable devices, in which the two steps of the extraction or the washing are preferably carried out, comprise mixer-settler apparatuses and extraction columns in the embodiments known to those skilled in the art.

The mixing according to step a) of the method according to the invention is customarily carried out in such a manner that the mixture A and the phase A (PA), after mixing, are in each case present in solution 1 at portions of 50 to 90% by weight of phase A (PA) and 10-50% by weight of mixture A. Preferably, the portions of phase A (PA) after mixing are between 60 and 80% by weight and the portions of mixture A are between 20 and 40% by weight. Particularly preferably, the portion of mixture A of solution 1 after step a) of the method according to the invention is about 25% by weight, and the portion of phase A (PA) of solution 1 is about 75% by weight. Solution 1 therefore forms a homogeneous mixed phase of mixture A with phase A (PA).

Preferably, in step b) the therapeutic protein (P) is extracted together with the low-molecular-weight impurity (N) from the phase A (PA) into the phase B (PB) of the solution b1 and the high-molecular-weight impurity (H) essentially remains in the phase A (PA) of the solution b2.

Solution b1 particularly preferably comprises greater than 90% by weight of the low-molecular-weight impurity (N) contained in the mixture A and greater than 90% by weight of the therapeutic protein (P) contained in mixture A. Very particularly preferably, solution b1 comprises greater than 99% by weight of the low-molecular-weight impurity (N) contained in mixture A and greater than 99% by weight of the therapeutic protein (P) contained in mixture A.

Solution b2 particularly preferably comprises less than 10% by weight of the therapeutic protein (P) contained in mixture A and greater than 90% by weight of the high-molecular-weight impurity (H) contained in mixture A. Very particularly preferably, solution b2 comprises less than 1% by weight of the therapeutic protein (P) contained in mixture A and greater than 99% by weight of the high-molecular-weight impurity (H) contained in mixture A.

Preferably, step c) of the method according to the invention is characterized in that a phase C (PC) is used which is employed in a combination with the phase B (PB) in which the distribution coefficient of the therapeutic protein (P) and the low-molecular-weight impurity (N) is of a value such that the therapeutic protein (P) is extracted from the phase B (PB) of the solution b1 into the phase C (PC) of the solution c1 and the low-molecular-weight impurity (N) remains as far as possible in the phase B (PB) of the solution c2.

This procedure is particularly advantageous, because by means of the change in the distribution coefficient, the therapeutic protein (P) can be further purified in a simple manner without a separate third phase which from one of the phases (PA, PC), which are similar to one another, needing to be used.

An alternative embodiment of the method according to the invention is characterized in that, in step c), an extraction is effected only by temperature change, wherein at least one thermosensitive polymer is used in the phase B (PB), in such a manner that in the context of step c), by temperature treatment of the solution b1, two phases form having differing solubilities for the therapeutic protein.

In the context of the alternative step c) of the novel method, then again two solutions c1 and c2 are formed having the properties stated hereinafter with respect to their content of therapeutic protein (P) and/or low-molecular-weight and/or high-molecular-weight impurity (H, N).

This alternative is particularly advantageous, because an extraction can thereby take place without a further phase C (PC).

Solution c1 particularly preferably comprises greater than 90% by weight of the therapeutic protein (P) contained in solution b1 and less than 40% by weight of the low-molecular-weight impurity (N) contained in solution b1.

Solution c2 particularly preferably comprises greater than 60% by weight of the low-molecular-weight impurity (N) contained in solution b1 and less than 10% by weight of the therapeutic protein (P) contained in solution b1.

Preferably, in step d), the therapeutic protein (P) is extracted from the phase A (PA) into the phase B (PB) of the solution d2, and the high-molecular-weight impurity (H) remains in the phase A (PA) of the solution d1.

Solution d1 particularly preferably comprises at least greater than 90% by weight of the high-molecular-weight impurity (H) contained in solution b2.

Solution d2 particularly preferably comprises at least greater than 90% by weight of the therapeutic protein (P) contained in solution b2.

Preferably, in step e), the therapeutic protein (P) remains in solution e1 comprising the phase C (PC), whereas the low-molecular-weight impurity (N) is extracted into the phase B (PB) of the solution e2.

Solution e1 therefore particularly preferably comprises at least greater than 90% by weight of the therapeutic protein (P) contained in solution c1.

Solution e2 therefore particularly preferably comprises at least greater than 90% by weight of the low-molecular-weight impurity (N) contained in solution c1.

The method according to the invention described here is distinguished, in particular, by achieving very high purities of the therapeutic protein (P), which is achieved by the succession of the extraction and wash steps. It has been particularly surprisingly found that the separate separation by extraction of the at least one high-molecular-weight impurity (H) and the further separation by extraction of the at least one low-molecular-weight impurity (N), together with the washing achieve the object particularly advantageously, since special devices are not required for any of the steps according to the invention that go beyond the devices for extraction known to those skilled in the art. Rather, all individual steps can be carried out in identical types of devices, since the method according to the invention can always be carried out by means of identical or similar phases.

These properties of the method according to the invention enable the preferred further developments of the method which are shown hereinafter.

A first preferred further development of the method according to the invention is characterized in that the solution d2 obtained from step d) is supplied together with phase B (PB) again to step b) of the method according to the invention. Particularly preferably, the phase B (PB) is only fed to step d) of the method according to the invention and solution d2 replaces the phase B (PB) of the step b) according to the invention.

This further development is advantageous because the yield of the therapeutic protein (P) from the method according to the invention is thereby increased, without additional expenditure in terms of apparatus resulting. In addition, the phase B (PB), contained in solution d2, can be used once again for the extraction, which in turn decreases the operating costs of a method operated in such a manner.

A further, likewise preferred further development of the method according to the invention is characterized in that, after step d) of the method according to the invention, in a step d*), the solution d1 obtained from step d) is subjected to an ultrafiltration or nanofiltration and optionally a reverse osmosis, in which the high-molecular-weight impurity (H) is separated from the phase A (PA) and this, optionally after addition of further salt for making up losses, is supplied together with the solution 1 again to step b) of the method according to the invention.

If a reverse osmosis takes place in the context of step d*), then the addition of further salt for making up losses is dispensed with, since the phase A (PA) can thereby be adapted again to the desired portion of the salts.

Not only the ultrafiltration or nanofiltration, but also the reverse osmosis are operated in this case in a manner generally known to those skilled in the art. In addition, it is known to those skilled in the art which devices are used for carrying out these methods.

The procedure of step d*) is particularly advantageous, because thereby, firstly the high-molecular-weight impurity (H) can be provided in very pure form in the phase A (PA) and this in some circumstances is likewise a valuable product, as described above, which increases the economic efficiency of the entire method.

An equally preferred further development of the method according to the invention is characterized by a step c*) in the form of a further extraction of the solution c2 resulting from step c) using the phase C (PC), or a phase D (PD) essentially similar to phase C, obtaining a solution c*1, comprising at least a portion of the phase C or D (PC, PD) and at least a portion of the therapeutic protein (P), and also obtaining a solution c*2, comprising at least a portion of the phase B (PB) and at least a portion of the low-molecular-weight impurity (N).

Preferably, in step c*), the remaining therapeutic protein (P) is extracted from the phase B (PB) into the phase C or D (PC, PD) of the solution c*1 and the low-molecular-weight impurity (N) remains in the phase B (PB) of the solution c*2.

Solution c*1 particularly preferably comprises greater than 90% by weight of the therapeutic protein (P) contained in solution c2. Very particularly preferably, solution c*1 comprises greater than 99% by weight of the therapeutic protein (P) contained in solution c2.

Solution c*2 particularly preferably comprises greater than 90% by weight of the low-molecular-weight impurity (N) contained in solution c2. Very particularly preferably, solution c*2 comprises greater than 99% by weight of the low-molecular-weight impurity (N) contained in solution c2.

Carrying out step c*) is preferred, because thereby the low-molecular-weight impurity (N) can be provided in very pure form in the phase B (PB) and, in some circumstances, this is likewise a valuable product, as described above, which increases the economic efficiency of the entire method.

An equally preferred further development of the method according to the invention is characterized by a step e*) in the form of a further extraction of the solution e2 resulting from step e) using the phase C (PC), or a phase E (PE) essentially similar to phase C, obtaining a solution e*1, comprising at least a portion of the supplied phase C or E (PC, PE) and at least a portion of the therapeutic protein (P), and also obtaining a solution e*2, comprising at least a portion of the phase B (PB) and at least a portion of the low-molecular-weight impurity (N).

Preferably the therapeutic protein (P) in step e*) remains in solution e*1 comprising the phase C or E (PC, PE), whereas the low-molecular-weight impurity (N) is extracted into the phase B (PB) of the solution e*2.

Solution e*1 particularly preferably comprises at least greater than 90% by weight of the therapeutic protein (P) contained in solution e2.

Solution e*2 particularly preferably comprises at least greater than 90% by weight of the low-molecular-weight impurity (N) contained in solution e2.

Carrying out step e*) is preferred, because thereby the low-molecular-weight impurity (N) can be provided in very pure form in phase B (PB) and this, in some circumstances, is likewise a valuable product as described above, which increases the economic efficiency of the entire method.

In a particularly preferred further development of the method according to the invention, the steps c*) and e*) are carried out and solution c*1 is combined with solution e*1 and this in turn is used either together with phase C (PC) supplied to step c) or used instead of this in step c) of the method according to the invention.

In a likewise particularly preferred further development of the method according to the invention, the steps c*) and e*) are carried out and solution c*2 and solution e*2 are combined and either supplied together with the phase B (PB) to step e) or used instead of phase B (PB) in step e) of the method according to the invention.

This further development is particularly advantageous, because thereby phase B (PB) can be used repeatedly in the entire method, and so the operating costs of the method can thus be reduced.

Very particularly preferably, solution c*2 and solution e*2 are supplied to step e) instead of phase B (PB), wherein optionally the combined solution of the solutions solution c*2 and solution e*2 is subjected to a further treatment according to a step f).

If a step f) is carried out, this can comprise an extraction of the low-molecular-weight impurity (N) from the combination of solution c*2 and solution e*2 using a phase F (PF), wherein the phase F (PF) comprises a thermosensitive polymer and the extraction is carried out according to the alternative embodiment of step c). Alternatively, the step f) can also comprise an evaporative crystallization and/or a crystallization and/or precipitation and/or ultrafiltration or nanofiltration in which the low-molecular-weight impurity (N) is separated off.

The further development of the method by a step f) is particularly advantageous, because thereby the low-molecular-weight impurity (N) can be provided in particularly high purity as a further by-product of the method according to the invention and the economic efficiency of the method thereby increased by sale of the same. In addition, the phase B (PB) can be recycled thereby and optionally reused in the method at another point, as a result of which the operating costs of the method are decreased.

If, as step f) of the preferred further development of the method according to the invention, an extraction is used, then this is preferably operated in such a manner as is already described in the preferred or alternative variants of the step c) according to the invention. Particularly preferably, in this case, the phase F (PF) used is recirculated with prior use of an ultrafiltration or nanofiltration for separating off the low-molecular-weight impurity (N).

The configuration of the step f) in the form just described is particularly advantageous, because the devices used for this are identical in type to the devices already used in the method according to the invention. Therefore, this configuration is particularly economical, since the devices have already been laid out for the method according to the invention.

In further preferred developments of the method, the solution e1 obtained from step e) can be subjected to a further purification step g), which is characterized in that it does not comprise an extraction according to the invention or a washing according to the invention.

This further purification step g) can comprise an ultrafiltration, nanofiltration or chromatographic method. The solution g obtained from the preferred purification step g) and comprising the phase C (PC) is particularly preferably combined with the solution c*1 and the solution e*1 and used together with the phase C (PC) supplied to step c), or instead of this phase C (PC), in step c) of the method according to the invention.

All process steps according to the invention and preferred variants thereof, and also the further developments and preferred variants thereof, can be carried out continuously or discontinuously. Preferably, all process steps are carried out continuously.

All process steps can also be carried out repeatedly in a similar form, without a further inventive concept being necessary. This applies equally to the further developments of the method.

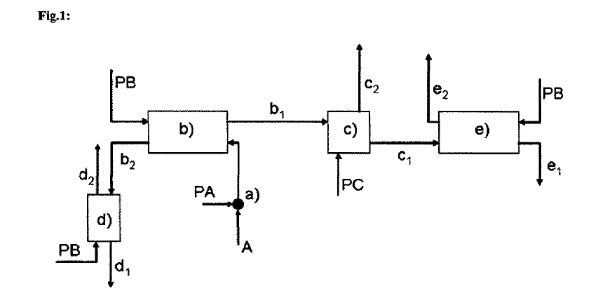

A special embodiment of the novel method is described in more detail with reference to FIG. 1, without restricting the invention thereto.

FIG. 1 shows how in a step a) the mixture A is combined with a phase A (PA) and first subjected to a countercurrent flow extraction b) using a second phase B (PB). Two phases b₁ and b₂, comprising solutions b₁ and b₂ result therefrom after settling. The solution b₁ is subjected to a further countercurrent flow extraction c) using a phase C (PC). Two phases c₁ and c₂ comprising solutions c₁ and c₂ result therefrom after settling. The solution c₁ is submitted to countercurrent flow washing using a further phase B (PB) according to a step e), from which, after settling, two phases e₁ and e₂ comprising solutions e₁ and e₂ result. The phase b₂ obtained from step b) and comprising the solution b₂ is subjected to a further countercurrent flow extraction d) using the phase B (PB) from which, after settling, the phases d₁ and d₂ comprising the solutions d₁ and d₂ result. The phase e₁ comprising the solution e₁ contains the high-purity therapeutic protein.

Hereinafter, special embodiments of the invention will be described with reference to examples, but without restricting the invention thereto.

### EXAMPLES

### Analytical Methods and Definitions

The total protein content in the respective salt solution or polymer solution is determined by a Bradford assay as per the scientific publication by MM. Bradford, Anal. Biochem 27 (1976) 248. In order to avoid effects due to solvent components, the samples are first diluted and analyzed against a blank sample.

The antibody concentration in both solutions is determined by protein A affinity chromatography on a Poros Protein A Column (Applied Biosystems, Foster City, Calif., USA). As binding buffer, a 10 mM sodium phosphate buffer containing 150 mM NaCl having a pH of 8.5 is used. The elution is performed using 12 mM HCl containing 150 mM NaCl. The absorption is observed at 280 nm. The samples of both phases are diluted in a sample buffer consisting of 0.05% by volume Tween80, 150 mM NaCl in 10 mM sodium phosphate buffer pH 8.5

The purity of both phases is determined by size exclusion chromatography (SEC) using a TSK-Gel Super SW3000 column (30 cm×4.6 mm I.D., 4 nm) from Tosoh Bioscience (Stuttgart, Germany). The samples, before analysis, are diluted at least tenfold with phosphate buffered saline (PBS). The analysis proceeds isocratically at 0.35 mL/min using a 50 mM phosphate buffer containing 300 mM NaCl at a wavelength of 215 nm.

In order to be able to evaluate the performance of the methods, varying parameters are used. The distribution coefficient K_{P} describes the concentration ratio of the protein in the respective phase of lower density in relation to a phase having the respectively higher density in a respective 2-phase system of individual steps. The protein yield in the respective phase having the lower density Y_{Top} is defined as the ratio of the antibody mass in this phase in relation to the entire antibody mass supplied to the system. The product purity in the respective phase having the lower density P_{SEC} is determined from the described SEC measurement as the quotient of the area of the IgG peak and the sum of all peaks in this phase.

In addition, the product purity is determined on the basis of the Bradford assay by P_{Bradford}. In this case, the purity is the ratio of the IgG concentration referred to the concentration of all protein components in the respective phase of lower density. The purification factor is reported as a percentage of the impurities removed.

### Example 1

A stock solution of 50% by weight of polyethylene glycol (PEG) having a median molecular weight of 3350 g/mol (Sigma St. Louis, Mo., USA) was produced in water. In addition, stock solutions of phosphate buffers consisting of anhydrous potassium hydrogenphosphate (K₂HPO₄), anhydrous sodium dihydrogenphosphate (NaH₂PO₄) and sodium chloride (NaCl) to 40% by weight in water, adjusted to various pHs according to Table 1, were produced.

TABLE 1
Composition of the stock solutions of
40% strength by weight phosphate buffer

| Buffer pH | NaH₂PO₄ (g) | K₂HPO₄ (g) | H₂O (g) |
|---|---|---|---|
| 6 | 28 | 22 | 75 |
| 7 | 16 | 34 | 75 |
| 8 | 6 | 44 | 75 |

For the experiments, human IgG for therapeutic applications (Gammanorm®Octapharma, Lachen, Switzerland) having a concentration of 165 mg/mL at a portion of 95% by weight of IgG of total protein content was used, and also a cell culture supernatant of a Chinese hamster ovary (CHO) cell culture having an IgG₁ against a human surface antigen (from Excellgene, Monthey, Switzerland). The latter was used at a concentration of 120 mg/L, based on the final volume and enriched with 0.5 g/L of Gammanorm®.

The aqueous two-phase systems were prepared by making up the polymer solution and the salt solution in each case separately from the stock solutions. The polymer solution and the salt solution formed the two phases.

The salt solution was prepared in such a manner that the required concentrations of NaCl and phosphate buffer were achieved after addition of the CHO cell supernatant. The CHO cell supernatant was added in a concentration of 25% by weight of the resultant salt solution.

The polymer solution was formed by PEG stock solution and a salt solution consisting of NaCl and phosphate and supplied to the method.

The salt solution and the polymer solution in the method together originally consisted of 8% by weight of PEG having a median molar mass of 3350 g/mol (PEG₃₃₅₀), 10% by weight of phosphate buffer having a pH of 6 and 10% by weight of NaCl.

The composition of the phases after adjusting the distribution equilibrium of the method corresponded in relation to PEG₃₃₅₀ and phosphate of the composition shown in Table 2 at a phase ratio of 0.4.

TABLE 2
Composition and physical properties of the
salt solution and the polymer solution

| Components/properties | Salt solution | Polymer solution |
|---|---|---|
| % (w/w) phosphates (PO₄³⁻) | 14.6 | 2.7 |
| % (w/w) PEG 3350 | 0.01 | 29.1 |
| % (w/w) Cl⁻ | 12.6 | 8.5 |
| ρ (Kg/m³) | 1236 | 1161 |
| μ (cp) | 2.8 | 27.0 |

For carrying out the experiment, a countercurrent mixer-settler battery (MSB) was used, consisting of an extraction comprising six extraction and settling zones, a back extraction comprising an extraction and settling zone, and a washing, comprising three extraction and settling zones.

13.5 kg of the salt solution (without the CHO cell supernatant) and 9 kg of the polymer solution were prepared. The separator was first half filled with the polymer solution. Then the salt solution and the CHO cell supernatant (enriched with Gammanorm, see above) were placed into the MSB.

As soon as the abovementioned salt solution had reached the third separator, the polymer solution, and also the salt solution for the light phase were run into the MSB in countercurrent flow. For the back extraction, 15 kg of a 14% strength by weight phosphate solution having pH 6 were prepared. The separator in the back-extraction step was half filled with this solution. As soon as the lighter phase (essentially polymer solution) had arrived from the extraction step in the mixer of the back-extraction step, the phosphate solution was transported into the same mixer. Then 20 kg of a 35% strength by weight PEG₃₃₅₀ solution were likewise prepared. All three separators of the wash stage were half filled therewith.

TABLE 3
Operating conditions of the MSB according to Example 1

| | F_{BP} | F_{CHO cell supernat} | F_{50% PEG 3350} | F_{salt sol.} | F_{14% phosphate} | F_{35% PEG 3350} |
|---|---|---|---|---|---|---|
| Step | (ml/h) | (ml/h) | (ml/h) | (ml/h) | (ml/h) | (ml/h) |
| Extraction | 450 | 195 | 202 | 131 | — | — |
| Back | — | — | — | — | 1011 | — |
| extraction | | | | | | |
| Wash | — | — | — | — | — | 1559 |

All process steps were run in the countercurrent flow process. The exact operating conditions of the individual process steps may be found in Table 3.

The results for purifying IgG from CHO cell supernatant are shown in Table 4.

TABLE 4
Characteristics of the purification of IgG according to Example 1

| | [IgG]_{Top/Bottom} | Y_{Top/Bottom} | P_{SEC} | Purification |
|---|---|---|---|---|
| Step | (mg/ml) | (%) | (%) | factor (%) |
| Start | — | — | 20 | — |
| Extraction | 0.28 | 89 | 26 | 42 |
| Back | 0.08 | 89 | 43 | 79 |
| extraction | | | | |
| Wash | 0.33 | 101 | 98 | 99.6 |

The advantages in particular of a multistage countercurrent procedure were demonstrated. A global yield of 80% and a final purity of 98% were achieved. At the same time, it may be derived therefrom that more than 99% of all minor components could be removed by means of the method.

This gave a total depletion of all minor components of greater than 99%. The therapeutic protein could be separated from its impurities by this method in typical extraction apparatuses and obtained in high purity and yield.

TABLE 5
Percentage portion of the removed impurities of the CHO cell supernatant
Removal of the components of the CHO cell supernatant (%)

| Step | 5.5 min* | IgG | 9.8 min* | 11.5 min* | 11.9 min* | 13.1 min* | 13.6 min* | 14.5 min* | 16.3 min* | 17.2 min* |
|---|---|---|---|---|---|---|---|---|---|---|
| Extraction | 100 | — | 67 | 65 | 32 | 26 | 0 | 0 | 0 | 0 |
| Back | 100 | — | 76 | 100 | 47 | 100 | 59 | 100 | 100 | 100 |
| extraction | | | | | | | | | | |
| Wash | 100 | — | 85 | 100 | 100 | 100 | 100 | 100 | 100 | 100 |

(*retention time of impurities in chromatographic analysis)

### Example 2

In a departure from Example 1, ten instead of six extraction and settling zones were used for the process step of the extraction. Furthermore, the concentration of IgG was increased to 1.5 g/L in order to demonstrate that the method can also be used for fermentation supernatants having high product concentrations. The exact operating conditions are given in Table 6. The solutions used were made up in a similar manner to Example 1.

TABLE 6
Operating conditions for the MSB for purifying IgG

| | F_{BP} | F_{CHO cell supernat} | F_{50% PEG 3350} | F_{salt sol} | F_{14% phosphate} | F_{30% PEG 3350} |
|---|---|---|---|---|---|---|
| Step | (ml/h) | (ml/h) | (ml/h) | (ml/h) | (ml/h) | (ml/h) |
| Extraction | 410 | 185 | 205 | 130 | — | — |
| Back | — | — | — | — | 485 | — |
| extraction | | | | | | |
| Wash | — | — | — | — | — | 580 |

The results of the method according to Example 2 are shown in Table 7.

A global yield of 80% and a final purity of 99% were achieved. This means that here too more than 99% of the impurities could be removed.

A still higher yield could be achieved by subjecting the heavy phase (essentially the salt solution) from the first extraction step to a further extraction. In this case, the global yield increased to 85%.

TABLE 7
Characteristics of the purification of IgG according to Example 2

| | [IgG]_{Top/Bottom} | Y_{Top/Bottom} | P_{SEC} | P_{Bradford} | Sum removal | Purification factor |
|---|---|---|---|---|---|---|
| Step | (mg/ml) | (%) | (%) | (%) | (%) | (%) |
| Start | — | — | 41 | 78 | — | — |
| Extraction | 0.66 | 77 | 46 | 102 | 69 | 45 |
| Back | 0.57 | 102 | 64 | 100 | 74 | 73 |
| extraction | | | | | | |
| Wash | 1.07 | 102 | 99 | 101 | 75 | 99.5 |

A majority of the high-molecular-weight impurities was already separated off during the first extraction step (see Table 8). The low-molecular-weight impurities, in contrast, were first removed by back extraction or washing.

The total depletion of all minor components is more than 99%.

TABLE 8
Percentage portion of the removed impurities
Removal of the compounds of the CHO cell supernatant (%)

| Step | 5.5 min* | IgG | 9.5 min* | 11.6 min* | 13.0-13.9 min* | 16.4 min* | 17.9 min* |
|---|---|---|---|---|---|---|---|
| Extraction | 100 | — | 77 | 44 | 7 | 0 | 0 |
| Back extraction | 100 | — | 79 | 70 | 74 | 100 | 100 |
| Wash | 100 | — | 84 | 100 | 100 | 100 | 100 |

(*retention time of impurities in chromatographic analysis)

Subsequently to the wash, the resultant solution was diafiltered. For this purpose, the heavy phase (essentially the salt solution) from the wash was concentrated batchwise and diafiltered in PBS buffer.

A laboratory crossflow filtration system, as is commercially available, was used (e.g. Sartoflow® Slice 200 benchtop crossflow system). The membrane used had a cutoff of 30 kDa with a membrane area of 200 cm² per cassette. Three cassettes were used in parallel, and so an active membrane area of 600 cm² was obtained.

The starting volume was 900 mL. During the diafiltration the original heavy phase (essentially the salt solution) was exchanged for PBS buffer. The exchange factor was approximately seven (900 mL of starting solution against 6900 mL of PBS buffer).

The temperature was held constant at 22-24° C. and the flow rate through the filter was set to 450 mL/min.

The main component was retained, whereas the salts (detected via the conductivity) were extracted by washing.

During the entire experiment, no IgG was found in the permeate.

It was therefore demonstrated that it is possible to exchange the buffer and to concentrate the antibody by a factor of 5.87 without loss of yield. Phosphate and also PEG were quantitatively removed.

1. A method for purifying therapeutic proteins starting from a mixture A, which method comprises at least one therapeutic protein (P), at least one high-molecular-weight impurity (H) and at least one low-molecular-weight impurity (N), comprising the steps of:
a. mixing the mixture A with a phase A (PA), obtaining a solution 1,
b. extracting the solution 1 resulting from step a), using a phase B (PB), obtaining a solution b1 comprising a portion of the phase B (PB), at least a portion of the therapeutic protein (P) and at least a portion of the low-molecular-weight impurity (N), and also obtaining a solution b2 comprising a portion of the phase A (PA), at least a portion of the high-molecular-weight impurity (H) and optionally a portion of the therapeutic protein (P),
c. further extracting the solution b1 resulting from step b), using a phase C (PC), obtaining a solution c1, comprising at least a portion of the supplied phase C (PC), at least a portion of the therapeutic protein (P) and optionally a portion of the low-molecular-weight impurity (N), and also obtaining a solution c2, comprising at least a portion of the phase B (PB) supplied with solution b1, at least a portion of the low-molecular-weight impurity (N) and optionally a portion of the therapeutic protein (P),
d. further extracting the solution b2 resulting from step b) using further phase B (PB), obtaining a solution d1 comprising at least a portion of the phase A (PA) supplied with solution b2 and at least a portion of the high-molecular-weight impurity (H), and also obtaining a solution d2 comprising at least a portion of phase B (PB) and optionally a portion of the therapeutic protein (P),
e. washing the solution c1 resulting from step c) using further phase B (PB), obtaining the one solution e1, comprising at least a portion of the phase C (PC), at least a portion of the therapeutic protein (P), optionally a portion of the low-molecular-weight impurity (N), and also obtaining a solution e2, comprising at least a portion of the phase B (PB), at least a portion of the low-molecular-weight impurity (N) and optionally a portion of the therapeutic protein (P).

2. The method as claimed in claim 1, wherein the therapeutic protein (P) is a protein that is useful used in medical procedures on humans.

3. The method as claimed in claim 1, wherein the high-molecular-weight, or the low-molecular-weight impurity (H, N) is insulin, growth hormones, albumins, interleukins, interferons, DNA, lectin, erythropoietin, glucose, lactate or amino acids.

4. The method as claimed in claim 1, wherein mixture A contains organisms or other solids.

5. The method as claimed in claim 1, wherein the phases A (PA) and C (PC) are solutions of at least one salt and/or at least one polymer in water.

6. The method as claimed in claim 1, wherein the phase B (PB) comprises a solution of at least one polymer, and said at least one polymer is soluble in water at least up to a portion of 10% by weight.

7. The method as claimed in claim 1, wherein, in step c), an extraction is effected only by temperature change, wherein at least one thermosensitive polymer is present in phase B (PB) of solution b1.

8. The method as claimed in claim 1, wherein the solution d2 obtained from step d) is supplied to step b) together with the phase B (PB) or instead thereof.

9. The method as claimed in claim 1, wherein, after step d), the solution d1 is subjected to an ultrafiltration or nanofiltration and optionally to reverse osmosis.

10. The method as claimed in claim 1, wherein the solution c2 is subjected to a further extraction, obtaining a solution c*1 and solution c*2.

11. The method as claimed in claim 1, wherein the solution e2 is subjected to a further extraction, obtaining a solution e*1 and solution e*2.

12. The method as claimed in claim 1, wherein the solutions c*2 and e*2 are combined and are supplied to step e) together with/instead of phase B (PB), wherein they are optionally subjected in advance to a further treatment.

13. The method as claimed in claim 12, wherein said further treatment is an extraction of the low-molecular-weight impurity (N) using a phase F (PF) comprising a thermosensitive polymer, or an evaporative crystallization and/or a crystallization and/or precipitation and/or ultrafiltration or nanofiltration.

14. The method as claimed in claim 1, wherein the solution e1 is subjected to a purification step obtaining a solution g, and wherein the purification step comprises an ultrafiltration, a nanofiltration or a chromatographic method.

15. The method as claimed in claim 14, wherein solution g is combined with the solutions c*1 and solution e*1 and supplied to step c) together with or instead of phase C (PC).

16. The method as claimed in claim 1, wherein each extraction and said washing is carried out in mixer-settler devices, comprising at least one extraction/wash zone with countercurrent flow and at least one settling zone.
